(19) United States
(12) Patent Application Publication
(10) Pub. No.: US 2022/0305071 A1
(43) Pub. Date: Sep. 29, 2022

(54) COMPOSITION COMPRISING PRUNUS PERSICA EXTRACT

(71) Applicant: OKCHUNDANG CO., LTD., Daegu (KR)

(72) Inventors: Chul Jong JUNG, Daegu (KR); Seok Man PARK, Daegu (KR); Gyung Yeun BEIK, Daegu (KR); Yoeng Eun YU, Daegu (KR)

(21) Appl. No.: 17/699,348

(22) Filed: Mar. 21, 2022

(30) Foreign Application Priority Data
Mar. 25, 2021 (KR) 10-2021-0038616

(51) Int. Cl. A61K 36/736; A61P 31/02

(52) U.S. Cl. CPC A61K 36/736; A61P 31/02; A61K 2236/333; A61K 2236/51

(57) ABSTRACT

Disclosed herein is a composition comprising a Prunus persica extract for skin wrinkle reduction, anti-oxidation, skin regeneration, skin whitening, or wound healing. Exhibiting high effects of skin wrinkle reduction antioxidation, skin regeneration, skin whitening, or wound healing, the Prunus persica extract can find various applications in cosmetic products for skin wrinkle reduction, antioxidation, skin regeneration, skin whitening and medicinal topical formulations for wound healing.

### BACKGROUND OF THE INVENTION

### 1. Field of the Invention

The present disclosure relates to a composition comprising a Prunus persica extract for skin wrinkle reduction, anti-oxidation, skin regeneration, skin whitening, or wound healing, and a preparation method therefor and, more specifically, to a composition comprising a crude solvent extract of Prunus persica or a solvent fraction thereof as an active ingredient for skin wrinkle reduction antioxidation, skin regeneration, skin whitening, or wound healing and a preparation method therefor. A Prunus persica extract according to the present disclosure can be used in a cosmetic composition for skin wrinkle reduction, anti-aging through antioxidation, skin whitening, and skin regeneration or in a pharmaceutical composition useful for treatment of wounds.

### 2. Description of the Prior Art

The skin is an organ that carries out an immune response while performing an essential barrier function to retain water inside the body and defend against external factors (antigens, pathogens, etc.).

With age, the human skin undergoes changes caused by various factors. The change factors may be classified into internal factors such as decreased in vivo hormone secretion levels, poor immune cell functions, etc. and external factors such as ultraviolet light, air pollution, contact with harmful substances, etc.

The external factors incur various troubles on the skin. Among them is skin damage. For the skin damage by skin damage inducer factors, damage occurs in the stratum corneum and fat layers, leading to transepidermal water loss, dry skin, wrinkle generation, itchiness, and inflammation by bacterial infection. Specifically, external stimuli incite keratinocytes in the epidermal basal layer to release various cytokines starting from IL-1a to IL-6, IL-8, TNF-a, etc. These cytokines provoke irritation and mediate topical inflammatory responses in the skin. Delayed restoration of the stratum corneum due to external stimuli such as continuous exposure to UV light might accelerate skin aging, resulting in various skin diseases. That is, the promotion of skin regeneration through normal differentiation and growth induction of keratinocytes can alleviate symptoms such as skin dryness, skin aging, itchiness, and so on.

In addition, oxidative stress attributed to free radicals and reactive oxygen species generated by UV light destroy the antioxidative defense line in vivo and oxidize the main skin constituents (lipids, proteins, polysaccharides, and nucleic acids) to promote the senescence of cells and tissues. Particularly, when proteins are oxidized, constituents of skin connective tissues, such as collagen, hyaluronic acid, elastin, proteoglycan, fibronectin, etc., are cleaved to incur excessive inflammatory responses and a decrease of skin elasticity, which might be aggravated to the extent of DNA mutation, cancer incidence, and immune dysfunction. In order to prevent skin aging, skin damage needs to be avoided by scavenging free radicals generated by various factors. Already damaged cells also need to be revived through regeneration and proliferation by active metabolisms.

Skin colors of humans depend greatly on the content of melanin pigments in skin cells. With a higher content of melanin pigments, the skin appears darker. An extremely rare content of melanin pigments may result in symptoms such as vitiligo, etc. Melanin pigments are produced from tyrosine by tyrosinase in a specialized group of cells known as melanocytes present in the basement membrane. Melanin pigments protect the skin against excessive UV radiation, playing an important role in suppressing UV radiation-induced skin damage and incidence of skin cancer. However, excessive melanogenesis due to hormone changes with age may cause a cosmetic problem along skin pigmentation. For example, melasma or freckles result from hyperpigmentation caused by increased melanin, indicating that melanogenesis is stimulated or melanocytes increase in number.

As such, skin aging could cause various changes and lesions. In order to delay such lesions or make restoration from lesions, various topical agents and drugs have been developed and used.

A wound refers to destruction of the continuum of a normal tissue by physicochemical injury or bacterial infection in the skin composed of the epidermis and dermis. Restoration from injured tissues is a complicate process that induces the restoration of injured skin or tissues to a normal state by a systematic biochemical cell metabolism responsible for the growth and regeneration of the tissues, with the concomitant induction of interactions among blood cells, cytokines, and growth factors. Treatment upon initial incidence is critical. Longer external exposure of an injured site is more prone to secondary infection which leads to the occurrence of complications. Thus, the injured site should be closed as early as possible. Various studies are ongoing into development of natural materials into medicinal or cosmetic substances exhibiting excellent efficacy without side effects. Some papers have recently verified the efficacy of Lespedeza cuneata extracts on wound healing and skin regeneration, reporting that composite formulations of extracts from Forsythia suspensa, Ulmus parvifolia Jacq., Canthium parviflorum, Lithospermi radix, and Gardeniae fructus would have healing effects. There were also reports on natural materials that are effective for treating the inflammatory responses that occur as inflammation-related cytokines are expressed by oxidative stress factors generated in the wound healing process.

With the increase of interest in physiologically active substances from plants, extensive studies have been conducted on the antioxidant, antibacterial, antimicrobial, anti-inflammatory, and anti-cancer effects of plant extracts, but their efficacy against wounds is evaluated as insignificant.

Peach (Prunus persica L. Batsch), which is one of representative Summer fruits, belongs to the genus Prunus in the rose family (Rosaceae) and is classified in the subgenus Amygdalus. Peach is composed mainly of water and sugars, with about 1% of organic acids such as tartaric acid, malic acid, citric acid, etc. The plant is abundant in esterified alcohols such as vitamin A, acetic acid, valeric acid, etc., aldehydes, pectin, and contains gum, benzoic acid, etc. in the xylem thereof and as such, has been used as a folk remedy for treating toothache. Because the glycosides, naringenin, quinic acid, lycopene, and tannin, and a small amount of nitrile glycoside found in the leaves exhibit anti-oxidative, anti-bacterial, and anti-inflammatory activities, a decoction of dry leaves or a mash of fresh leaves has been reported to exhibit the effect of relieving heart rash or eczema or diminishing small furuncles when topically applied or orally administered. In the Song Dynasty of China, fumigation methods using peach leaves are recorded for the purpose of treating or preventing “celestial diseases” (infectious diseases). In addition, Dong-ui-bo-gam discloses that peach leaves are used to treat the itchiness and pain like a bug's bite, in the loose vulva. According to the Encyclopedia of Chinese Herbal Medicine ( [symbol] ), peach laves are recorded to be used as a purgative agent or an anthelmintic and to treat malaria and trichomoniasis. Although most of the studies into peach pertain to peach kernels or flesh, there are reports on research about chemical ingredients and biological activities of peach leaves and stems, anti-oxidative activity and effect of peach leaf extract-mixed soap and packs on change in skin state of men, assay for functionality of a peach leaf extract as a natural cosmetic material, etc. However, research on peach leaves is still scarce.

### SUMMARY OF THE INVENTION

In the present disclosure, a cosmetic composition for skin wrinkle reduction, anti-oxidation, skin regeneration, and skin whitening, and a composition for wound healing, each comprising a Prunus persica extract, were prepared, and identified to have excellent effects of wrinkle reduction, anti-oxidation activity, skin regeneration, and wound healing.

Accordingly, an aspect of the present disclosure is to provide a pharmaceutical composition comprising a Prunus persica extract as an active ingredient for wound healing.

Another aspect of the present disclosure is to provide a method for preparing a pharmaceutical composition comprising a Prunus persica extract as an active ingredient for wound healing.

A further aspect of the present disclosure is to provide a cosmetic composition comprising a Prunus persica extract as an active ingredient for promoting skin regeneration.

A still further aspect of the present disclosure is to provide a cosmetic composition comprising a Prunus persica extract as an active ingredient for skin wrinkle reduction.

Still another aspect of the present disclosure is to provide a cosmetic composition comprising a Prunus persica extract as an active ingredient for increasing anti-oxidative activity on the skin.

Yet another aspect of the present disclosure is to provide a cosmetic composition comprising a Prunus persica extract as an active ingredient for skin whitening.

The present disclosure relates to a composition comprising a Prunus persica extract for skin wrinkle reduction, anti-oxidation, skin regeneration, skin whitening, or wound healing. The Prunus persica extract according to the present disclosure has excellent skin wrinkle reduction, anti-oxidation, skin regeneration, and skin whitening effects and exhibits a high therapeutic effect on wounds.

In the present disclosure, the Prunus persica extract was examined for the effects of skin wrinkle reduction, anti-oxidation, skin regeneration, skin whitening, and wound healing.

### BRIEF DESCRIPTION OF THE DRAWINGS

The above and other aspects, features and advantages of the present disclosure will be more apparent from the following detailed description taken in conjunction with the accompanying drawings, in which:

FIG. 1 is a flow scheme illustrating the preparation of a Prunus persica leaf water extract and a Prunus persica leaf prethanol extract according to an embodiment of the present disclosure;

FIG. 2 is a graph showing DPPH radical scavenging activity of Prunus persica leaf extracts by concentrations according to an embodiment of the present disclosure;

FIG. 3 is a graph showing ABTS radical scavenging activity of Prunus persica leaf extracts by concentration according to an embodiment of the present disclosure;

FIG. 4 is a plot of reducing power of Prunus persica leaf extracts against concentrations according to an embodiment of the present disclosure;

FIG. 5 is a graph showing collagenase inhibition rates of Prunus persica leaf extracts by concentration according to an embodiment of the present disclosure;

FIG. 6 is a graph showing tyrosinase inhibition rates of Prunus persica leaf extracts by concentration according to an embodiment of the present disclosure;

FIG. 7 is a graph showing cell viability of macrophages (Raw 264.7) by concentrations Prunus persica leaf extracts according to an embodiment of the present disclosure;

FIG. 8 is a graph showing nitric oxide inhibition activity of Prunus persica leaf extracts by concentration according to an embodiment of the present disclosure;

FIG. 9 is a graph showing would healing effects and skin regeneration effects of Prunus persica leaf extracts as measured by a wound healing assay (Con: non-treated, Cta: Centella asiatica extract, PFE: Prunus persica leaf extract) according to an embodiment of the present disclosure;

FIG. 10 shows photographic images illustrating wound healing and re-epithelialization effects of Prunus persica leaf extracts as examined by a wound healing assay (Con: non-treated, Cta: Centella asiatica extract, PPL: Prunus persica leaf extract) according to an embodiment of the present disclosure;

FIG. 11 shows digital camera images of wound sites day 0, 3, and 7 after treatment without (NO) or with Madecasol (MC), 10 μL of the Prunus persica leaf extract (PFE10), and 100 μL of the Prunus persica leaf extract (PFE100) according to an embodiment of the present disclosure;

FIG. 12 shows images illustrating re-epithelization effects as assayed by immunohistochemical staining after wound sites of rats were treated without (NO) or with Madecasol (MC), 10 μL of the Prunus persica leaf extract (PFE10), and 100 μL of the Prunus persica leaf extract (PFE100) according to an embodiment of the present disclosure;

FIG. 13 is a graph showing re-epithelization as quantitatively assayed by immunohistochemical staining after wound sites of rats were treated without (NO) or with Madecasol (MC), 10 μL of the Prunus persica leaf extract (PFE10), and 100 μL of the Prunus persica leaf extract (PFE100) according to an embodiment of the present disclosure (*p<0.05, **p<0.01 and ^{#}p<0.001);

FIG. 14 shows images showing re-epithelization as assayed by Masson's trichrome stain after wound sites of rats were treated without (NO) or with Madecasol (MC), 10 μL of the Prunus persica leaf extract (PFE10), and 100 μL of the Prunus persica leaf extract (PFE100) according to an embodiment of the present disclosure; and

FIG. 15 is a graph showing re-epithelialization as quantitatively assayed by Masson's trichrome stain after wound sites of rats were treated without (NO) or with Madecasol (MC), 10 μL of the Prunus persica leaf extract (PFE10), and 100 μL of the Prunus persica leaf extract (PFE100) according to an embodiment of the present disclosure (*p<0.05, **p<0.01 and ^{#}p<0.001).

### DETAILED DESCRIPTION OF THE EXEMPLARY EMBODIMENTS

The present disclosure pertains to a method for treating or relieving wound, the method comprising the following step of:

applying to an affected area a composition comprising a Prunus persica extract as an active ingredient.

Below, a detailed description will be given of the present disclosure.

As used herein, the term “Prunus persica” refers to a tree belonging to the subgenus Amygdalus in the family Rosaceae and native to plateaus 600-2,000 m above sea level in Shanxi Province and Gansu Province in Hwabuk, China. Generally, Prunus persica is called a peach tree, as is a small deciduous tree.

An aspect of the present disclosure pertains to a pharmaceutical composition comprising a Prunus persica extract as an active ingredient for wound healing.

As used herein, the term “extract” is intended to encompass a solvent crude extract, an extract dissolved in a specific solvent (solvent fraction), and a solvent fraction of a solvent crude extract, and the Prunus persica extract may be in a solution, concentrate, or powder state.

The Prunus persica extract according to the present disclosure may be obtained as an extract from at least one selected from the group consisting of leaves, stems, fruits, and roots of Prunus persica and, for example, an extract from leaves of Prunus persica, but with no limitations thereto.

In the present disclosure, the Prunus persica extract may be a crude extract obtained by extracting at least one selected from the group consisting of leaves, stems, fruits, and roots of Prunus persica in at least one solvent selected from the group consisting of water, a straight or branched alcohol of 1 to 4 carbon atoms, and prethanol and, for example, an extract obtained by extracting in prethanol, but with no limitations thereto.

As used herein, the term “prethanol” refers to vegetable ethanol used as a food additive, and throughout this specification, the term “prethanol” is used interchangeably with the term “ethanol”.

In an embodiment of the present disclosure, the Prunus persica extract may be an extract obtained by extracting at least one selected from the group consisting of leaves, stems, fruits, and roots of Prunus persica in prethanol having an alcohol content of 10% (inclusive) to 100% (v/v) (exclusive), 20% (inclusive) to 100% (v/v) (exclusive), 30% (inclusive) to 100% (v/v) (exclusive), % (inclusive) to 100% (v/v) (exclusive), 50% (inclusive) to 100% (v/v) (exclusive), 60% (inclusive) to 100% (v/v) (exclusive), or 70% (inclusive) to 100% (v/v) (exclusive), 10% to 60% (v/v), 20% to 60% (v/v), 10% to 50% (v/v), 10% to 40% (v/v), 20% (inclusive) to 50% (v/v), 20% to 45% (v/v), or 25% to 35% (v/v).

When a mixture of water and alcohol is used as a solvent in preparing a crude extract of Prunus persica, the solvent may be an aqueous alcohol solution contains a straight or branched alcohol of 1 to 4 carbon atoms at a concentration of 10% (inclusive) to 100% (v/v) (exclusive), 20% (inclusive) to 100% (v/v) (exclusive), 30% (inclusive) to 100% (v/v) (exclusive), 40% (inclusive) to 100% (v/v) (exclusive), 50% (inclusive) to 100% (v/v) (exclusive), 60% (inclusive) to 100% (v/v) (exclusive), or 70% (inclusive) to 100% (v/v) (exclusive).

The aqueous alcohol solution may be at least one selected from the group consisting of an aqueous methanol solution, an aqueous ethanol solution, an aqueous propanol solution, and an aqueous butanol solution.

The Prunus persica extract according to the present disclosure may be a solvent fraction obtained by fractionating the solvent crude extract with an additional solvent and, for example, a solvent fraction obtained with a polar solvent such as water, an alcohol, ethylene glycol, butylene glycol, a lower alcohol of 1 to 4 carbon atoms, or a non-polar solvent such as hexane, ethyl acetate, chloroform, dichloromethane, alone or in combination, but with no limitations thereto.

The preparation of the Prunus persica extract according to the present disclosure will be elucidated in detail as follows: Prunus persica leaves are cleansed and completely dried before being ground for easy extraction. Extraction is made using an extraction solvent in an amount of about 5 to 20 volumes and preferably 7 to 15 volumes relative to the weight of the ground Prunus persica leaves. Following extraction, filtration, vacuum concentration, and lyophilization are conducted sequentially. Although no particular limitations are imparted to the extraction temperature, extraction may be conducted at 40 to 110° C. or 55 to 90° C. when using water as a solvent and at 10 to 40° C. when using prethanol as a solvent.

The extraction process may be conducted once or repeated many times. In an embodiment of the present disclosure, re-extraction may be conducted after primary extraction. Even if the filtration is effectively conducted, there must be a loss because medicinal herbs have high water contents. Thus, a poor extraction efficiency is obtained with primary extraction alone. In order to prevent such poor efficiency, extraction may be repeated. In addition, investigation data for extraction efficiency in each step indicated that 80 to 90% of the total extract was obtained by the first two rounds of extraction.

Upon preparation of the Prunus persica extract, too small an amount of a solvent makes the stirring difficult and lowers the solubility of the extract, resulting in a decrease in extraction efficiency. When the solvent is excessively used, the amount of the solvent to be filtered in the next step is a burden in terms of handling and thus is economically unfavorable. Therefore, the amount of the solvent may be preferably in the range stated above, but is not limited thereto.

Any conventional extraction method may be used in the present disclosure and examples of the extraction method include cold precipitation, hot-water extraction, ultrasonic extraction, and reflux cold extraction, but are not limited thereto.

The present disclosure provides a composition comprising a Prunus persica extract as an active ingredient for wound healing. The Prunus persica extract may exist as a solvent crude extract of Prunus persica or a solvent fraction thereof as described above.

The wound may be at least one selected from the group consisting of a burn, a laceration, epidermal wound, ulcer, trauma, post-surgical wound, wound generated upon delivery, chronic wound, and injuries caused by dermatitis, corneal ulcer, corneal epithelial detachment, keratitis, and dry eye syndrome.

The content of the extract as an active ingredient in the composition according to the present disclosure may be properly adjusted depending on the form and purpose of use, patient's conditions, types and severity of syndrome and may range from 0.001 to 99.9% by weight, or 0.1 to 99.9% by weight, for example, 0.1 to 50% by weight or 0.1 to 40% by weight, based on the weight of solid content, but is not limited.

The composition according to the present disclosure may be administered to mammals including humans via various routes. Any conventional administration mode may be contemplated. Examples of the administration routes include oral, dermal, intravenous, intramuscular, and subcutaneous routes. For instance, the composition may be administered via a dermal route. The composition of the present disclosure may be formulated into oral dosage forms such as pulvis, granules, tablets, capsules, ointment, suspensions, emulsions, syrups, aerosols, and so on, or parenteral dosage forms such as percutaneous agents, suppositories, and sterile injections.

In an embodiment of the present disclosure, the extract may be formulated into an ointment, for example, an ointment encapsulated with a liposomal membrane.

The composition of the present disclosure may contain a proper and physiologically acceptable auxiliary agent such as a carrier, an excipient, and a diluent, in addition to the mixed extract.

Examples of the carrier, excipient, and diluent available for the composition of the present disclosure include lactose, dextrose, sucrose, sorbitol, mannitol, erythritol, maltitol, xylitol, erythritol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

The composition is formulated using conventional diluents or excipients, such as fillers, extenders, binders, wetting agents, disintegrants, and surfactants. Solid formulations for oral administration include tablets, pills, powders, granules, capsules, etc. These solid formulations may be prepared by mixing at least one compound with one or more excipients, for example, starch, calcium carbonate, sucrose, lactose, gelatin, etc. In addition to a simple excipient, a lubricant such as magnesium stearate, talc, etc. may be used.

Liquid formulations for oral administration include a suspension, a solution, an emulsion, a syrup, an ointment, etc. In addition to water commonly used as a simple diluent and liquid paraffin, various excipients, for example, wetting agents, sweetening agents, flavors, preservatives, etc. may be included.

Formulations for parenteral administration include sterilized aqueous solutions, non-aqueous solvents, suspending agents, emulsions, freeze-drying agents, suppositories, percutaneous agents, etc. Propylene glycol, polyethylene glycol, vegetable oils such as olive oil, injectable esters such as ethyl oleate, etc. may be used as non-aqueous solvents and suspending agents.

Bases for suppositories may include witepsol, macrogol, tween 61, cacao butter, laurin butter, glycerinated gelatin, etc.

When applied to humans according to an embodiment, the composition of the present disclosure may be administered alone or in mixture with a pharmaceutically acceptable carrier selected in consideration of general administration modes and the standard pharmaceutical practice.

For example, the composition containing the herb extract of the present disclosure may be orally or sublingually administered in a tablet form containing starch or lactose, a capsule form containing only the composition of the present invention or containing an excipient in addition to the composition, or an elixir or suspension form containing a chemical for flavor or color. A liquid formulation is prepared together with a pharmaceutically acceptable additive such as a suspending agent (for example, methylcellulose, semi-synthetic glycerides such as Witepsol, glyceride mixture such as a mixture of apricot kernel oil and polyethylene glycol (PEG)-6 ester or a mixture of PEG-8 and caprylic/capric glyceride).

The dose of the composition of the present disclosure may vary depending on various factors including the patient's age, weight, and sex, the mode of administration, health states, and disease severity, and it may be administered once to several times as divided a day in a certain interval according to the judgment of doctors or pharmacists.

For example, the daily dosage may be 0.1 to 500 mg/kg and preferably 0.5 to 300 mg/kg on the basis of content of active ingredient. The dosage is an example of average case and the dosage may be higher or lower according to the difference of individuals.

When the daily dosage of the composition according to the present disclosure is lower than the above administration dose, a significant effect cannot be obtained, and a dosage which is over the upper limit of the range is disadvantaged. A dose beyond the common range may cause an undesirable side effect, and therefore the above range is preferable.

Another aspect of the present disclosure pertains to a cosmetic composition comprising a Prunus persica extract as an active ingredient for promoting skin regeneration.

Since the cosmetic composition for promoting skin regeneration according to the present disclosure contains the same Prunus persica extract as in the aforementioned pharmaceutical composition for wound healing, the common content therebetween is omitted in order to avoid undue complexity of the specification

The cosmetic composition according to the present disclosure may be applied to at least one region selected from the group consisting of hair, scalp, skin, mouth, teeth, and periodontia.

When applied, the cosmetic composition according to the present disclosure may be prepared into any conventional formulation. For example, the composition may be formulated into a solution, an emulsion, a suspension, an oily solution, a cream, a paste, a gel, a beauty wash, a pack, a lotion, a powder, a spray, a soap, a soft lotion, a nutrient lotion, a nutrient essence, a nutrient oil, a hydration oil, a foundation, a makeup base, a cleanser, a shampoo, a lotion, and an ointment.

When applied to the hair or scalp, the cosmetic composition according to the present disclosure may be formulated into hair care products, such as hair toners, hair lotions, hair creams, hair sprays, hair mousses, hair gels, hair soaps, hair shampoos, hair rinses, hair packs, and hair treatments, but with no limitations thereto.

For the ointment, paste, cream, or gel formulated with the cosmetic composition of the present disclosure, animal oil, vegetable oil, wax, paraffin, starch, tragacanth, cellulose derivatives, polyethylene glycol, silicon, bentonite, silica, talc, zinc oxide, etc. may be used as a carrier, alone or in combination, but with no limitations thereto.

When the cosmetic composition of the present disclosure is formulated into a powder or spray, lactose, talc, silica, aluminum hydroxide, calcium silicate, polyamide powder, etc. may be used as a carrier. For example, a propellent such as chlorofluorohydrocarbon, propane/butane, or dimethyl ether, may be additionally included in a spray, but with no limitations thereto.

When the cosmetic composition of the present disclosure is formulated into a solution or emulsion, a solvent, a solubilizer, or an emulsifier may be available as a carrier. For example, water, ethanol, isopropanol, ethyl carbonate, ethyl acetate, benzyl alcohol, benzyl benzoate, propylene glycol, 1,3-butyl glycol oil, etc. may be employed. Examples of the carrier include cotton seed oil, peanut oil, corn germ oil, olive oil, castor oil, sesame oil, glycerol aliphatic ester, polyethylene glycol, fatty acid esters of sorbitan, but are not limited thereto.

When the cosmetic composition of the present disclosure is formulated into a suspension, a liquid diluent such as water, ethanol, or propylene glycol, a suspending agent such as ethoxylated isostearyl alcohol, polyoxyethylene sorbitol ester, and polyoxyethylene sorbitan ester, microcrystalline cellulose, aluminum hydroxide, bentonite, agar, or tragacanth may be employed as a carrier, but with no limitations thereto.

In the present disclosure, the cosmetic composition contains a Prunus persica extract in an amount of 0.001 to 30% by weight, based on the total weight thereof, but with no limitations thereto.

Another aspect of the present disclosure is concerned with a cosmetic composition comprising a Prunus persica extract as an active ingredient for reducing skin wrinkles.

Since the cosmetic composition for reducing skin wrinkles according to the present disclosure contains the same Prunus persica extract as in the aforementioned pharmaceutical composition for wound healing, the common content therebetween is omitted in order to avoid undue complexity of the specification.

Another aspect of the present disclosure pertains to a cosmetic composition comprising a Prunus persica extract as an active ingredient for increasing anti-oxidative activity on the skin.

As used herein, the term “anti-oxidative activity” refers to an action of inhibiting oxidation. There is a balance between pro-oxidants and anti-oxidants in the human body. However, when the balance collapses due to various factors to make a bias toward oxidation promotion, oxidative stress is induced in vivo, with the consequent onset of cell damages and pathological diseases. Reactive oxygen species (ROS), which is a direct cause of oxidative stress, is chemically unstable and highly reactive and easily reacts with various biomaterials such as DNA, proteins, lipids, and carbohydrates. ROS attacks biopolymers in vivo to cause irreversible damages in cells and tissues or to bring about mutation, cytotoxicity, and cancer, playing as a direct cause of senescence. Removal or diminution of ROS brings about an anti-oxidative effect which leads to anti-aging and health maintenance.

Since the cosmetic composition for increasing anti-oxidative activity on the skin according to the present disclosure contains the same Prunus persica extract as in the aforementioned pharmaceutical composition for wound healing, the common content therebetween is omitted in order to avoid undue complexity of the specification.

Another aspect of the present disclosure pertains to a cosmetic composition comprising a Prunus persica extract as an active ingredient for skin whitening.

The term “skin whitening”, as used herein, refers to brightening a skin tone by relieving or reducing deposition of various pigments, such as melasma, freckles, and so on, due to excessive synthesis of melanin, activation of tyrosinase, melanogenesis, etc. Through the skin whitening effect, skin tone or complexion can be improved.

Another aspect of the present disclosure is directed to a method for treating or relieving skin wound, the method comprising a step of applying to an affected area of a subject a composition comprising a Prunus persica extract as an active ingredient.

Another aspect of the present disclosure is directed to a method for promoting skin regeneration, the method comprising a step of applying to a skin of a subject a composition comprising a Prunus persica extract as an active ingredient.

Another aspect of the present disclosure is directed to a method for cosmetically treating a skin condition, the method comprising a step of applying to a target skin a composition comprising a Prunus persica extract as an active ingredient.

Since the cosmetic composition for skin whitening according to the present disclosure contains the same Prunus persica extract as in the pharmaceutical composition for wound healing and the cosmetic composition for promoting skin regeneration, the common content therebetween is omitted in order to avoid undue complexity of the specification.

A better understanding of the present disclosure may be obtained via the following examples which are set forth to illustrate, but are not to be construed as limiting the present disclosure.

### Example 1: Preparation of Prunus persica Extract

1-1. Preparation of Water Extract (PWE)

For use in experiments, Prunus persica leaves were taken from nectarine harvested August, 2018 from an orchard located at Jain myon, Kyeongsan gun, Gyeongsangbuk-do before the growth of the leaves was terminated. The Prunus persica leaves taken were selectively divided, washed, and completely dried in a shade place before being ground.

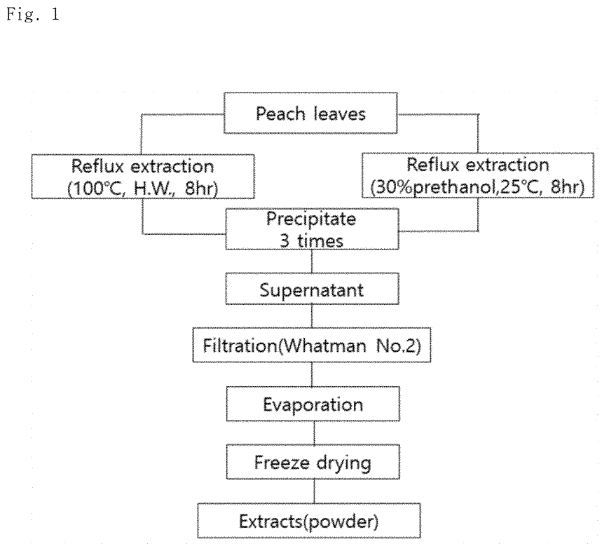

A Prunus persica water extract was prepared as illustrated in FIG. 1. In brief, the ground Prunus persica leaves were put, together with 10 volumes of hot water, into a round-bottom flask equipped with a reflux condenser and extraction was conducted three times, each for eight hours or longer, at 100° C. The extract solution thus obtained was filtered through filter paper (Whatman No. 2) and the filtrate was concentrated in a vacuum using a rotary vacuum evaporator (R-100, BÜCHI, Germany), followed by lyophilization in a freeze drier (TFD5505, ilShin BioBase, Korea). Then, the lyophilizate was used in subsequent experiments.

1-2. Preparation of Prethanol Extract (PFE)

For use in experiments, Prunus persica leaves were taken from nectarine harvested August, 2018 from an orchard located at Jain myon, Kyeongsan gun, Gyeongsangbuk-do before the growth of the leaves was terminated. The Prunus persica leaves taken were selectively divided, washed, and completely dried in a shade place before being ground.

A prethanol extract was prepared as illustrated in FIG. 1. In brief, the ground Prunus persica leaves were put, together with 10 volumes of 30% prethanol, into a round-bottom flask equipped with a reflux condenser and extraction was conducted three times, each for eight hours or longer, at 25° C. The extract solution thus obtained was filtered through filter paper (Whatman No. 2) and the filtrate was concentrated in a vacuum using a rotary vacuum evaporator (R-100, BÜCHI, Germany), followed by lyophilization in a freeze drier (TFD5505, ilShin BioBase, Korea). Then, the lyophilizate was used in subsequent experiments.

### Example 2: Assay for Polyphenol and Flavonoid Contents

2-1. Polyphenol Content

In order to measure polyphenol contents, 2 mL of 2% sodium carbonate and 100 μL of 50% Folin-Ciocalteu reagent were added to 100 μL of each sample extract solution, followed by reading absorbance at 720 nm. The total polyphenol contents were calculated using a standard calibration curve for the standard substance gallic acid and are given in Table 1, below.

TABLE 1

| | Total polyphenol content |
|---|---|
| | (mg GAE/g) |
| Primus persica water | 545.82 ± 0.55 |
| extract | |
| Prunus persica prethanol | 995.42 ± 0.78 |
| extract | |

As can be seen in Table 1, the total polyphenol contents were measured to be in the range of 545.82 to 995.420 mg GAE/g, with a higher level in the Prunus persica leaf prethanol extract (PFE) than in the Prunus persica leaf water extract (PWE).

2-2. Flavonoid Content

From a dilution of 100 μL of each sample solution in 900 μL of 80% ethanol, 100 μL was taken, mixed with 4.3 mL of 80% ethanol containing 10% aluminum nitrate and 1 M potassium acetate, and left at room temperature for 40 min followed by reading absorbance at 415 nm. In this regard, flavonoid contents were calculated using a standard calibration curve for gallic acid and are expressed as gallic acid equivalents (mg GAE/g extract) in Table 2, below.

TABLE 2

| | Total Flavonoid Content |
|---|---|
| | (mg GAE/g) |
| Prunus persica water | 34.50 ± 0.20 |
| extract | |
| Prunus persica prethanol | 46.70 ± 0.23 |
| extract | |

As can be seen in Table 2, the total flavonoid contents in the Prunus persica leaf extracts were measured to be in the range of 34.50 to 46.70 mg GAE/g, with a lower level in the Prunus persica leaf water extract (PWE) than in the Prunus persica leaf prethanol extract (PFE), like the polyphenol content.

### Example 3: Assay for Electron Donating Ability

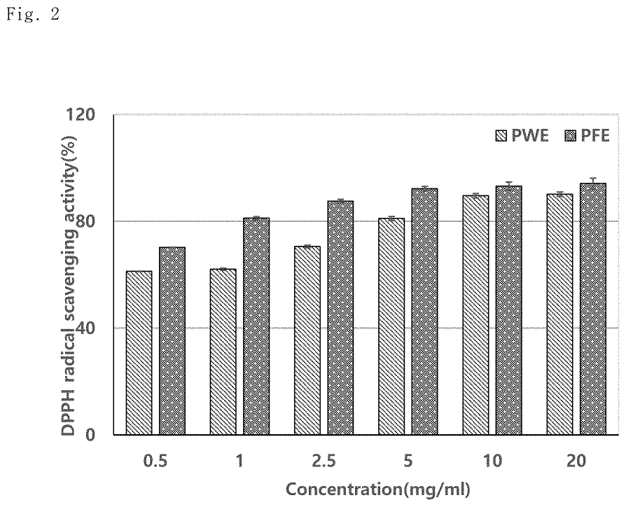

For assay for electron donating ability (EDA), extract solutions were uniformly diluted and 0.2 mL of each dilution was added with 0.8 mL of 0.4 mM DPPH (1,1 diphenyl-2-piery-hydrazyl), vortexed for 10 seconds, and left at room temperature for 10 minutes, followed by reading absorbance at 525 nm to measure DPPH radical scavenging activity through which the electron donating ability was assayed. The electron donating ability was calculated according to Equation 1, below, and the results are depicted in FIG. 2 and summarized in Table 3.

Electron donating ability (%)={1−(absorbance of sample added group/absorbance of non-added group)}×100 <Equation 1>

TABLE 3

| | Prunus persica water | | | | | | Prunus persica prethanol | | | | | |
|---|---|---|---|---|---|---|---|---|---|---|---|---|
| | extract (mg/ml) | | | | | | extract (mg/ml) | | | | | |
| | 0.5 | 1 | 2.5 | 5 | 10 | 20 | 0.5 | 1 | 2.5 | 5 | 10 | 20 |
| DPPH | 61.25 | 62.02 | 70.54 | 81.04 | 89.55 | 90.14 | 70.24 | 81.14 | 87.54 | 92.14 | 93.15 | 94.12 |
| Radical | | | | | | | | | | | | |
| scavenging | | | | | | | | | | | | |
| activity | | | | | | | | | | | | |
| (%) | | | | | | | | | | | | |

As can be seen in Table 3 and FIG. 2, the Prunus persica leaf water extract and the Prunus persica leaf prethanol extract were measured to have an EDA of 89.55% and 93.15%, respectively, at a concentration of 10 mg/mL, with superiority of the Prunus persica leaf prethanol extract to the water extract.

### Example 4: Assay for ABTS Radical Scavenging Activity

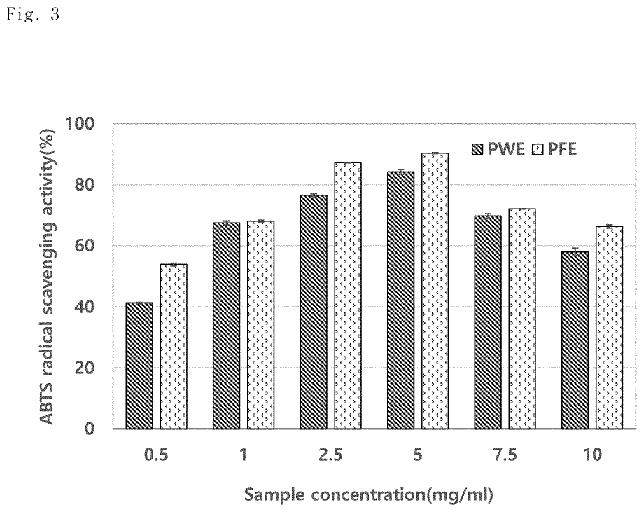

ABTS radical scavenging activity was assayed by a method based on the principle that ABTS radicals which react with potassium persulfate to produce a blue solution change in color from blue green to pale green in the presence of an antioxidant in the extract. A mixture of 7.4 mM 2, 2-azino-bis-(3-ethylbenzothiazoline-6-sulfonic acid)-diammonium salt and 2.4 mM potassium persulfate was allowed to stand in the dark at room temperature for 24 hours to produce ABTS+ cation radicals which were then diluted with 50% ethanol until the absorbance was 0.70±0.02 at 734 nm. Each extract was diluted by concentration and 50 μL of each dilution was added with 1 mL of the ABTS solution and incubated for 3 minutes. Absorbance was read at 734 nm on an ELISA reader (ECNSPIREMD, PerkinElmer, Germany). With L-ascorbic acid serving as a control, comparison was made of absorbance relative to the non-added group to calculate ABTS radical scavenging activity according to Equation 2, below. The data are summarized in Table 4 and depicted in FIG. 3.

ABTS radical scavenging activity (%)={1−(absorbance of sample added group/absorbance of non-added group)}×100 <Equation 2>

TABLE 4

| | Prunus persica water | | | | | | Prunus persica prethanol | | | | | |
|---|---|---|---|---|---|---|---|---|---|---|---|---|
| | extract (mg/ml) | | | | | | extract (mg/ml) | | | | | |
| | 0.5 | 1 | 2.5 | 5 | 7.5 | 10 | 0.5 | 1 | 2.5 | 5 | 7.5 | 10 |
| ABTS | 41.25 | 67.48 | 76.55 | 84.16 | 69.69 | 57.97 | 53.86 | 68.03 | 87.18 | 90.31 | 72.07 | 66.32 |
| radical | | | | | | | | | | | | |
| scavenging | | | | | | | | | | | | |
| activity | | | | | | | | | | | | |
| (%) | | | | | | | | | | | | |

As can be seen in Table 4 and FIG. 3, the ABTS radical scavenging activity was drastically increased in both PWE and PFE until the concentration of the Prunus persica extract reached 5 mg/mL, and then significantly decreased at higher concentrations. At 5 mg/mL, the ABTS radical scavenging activity peaked 84.16% for the Prunus persica leaf water extract and 90.31% for the Prunus persica leaf prethanol extract.

### Example 5: Assay for Reducing Power

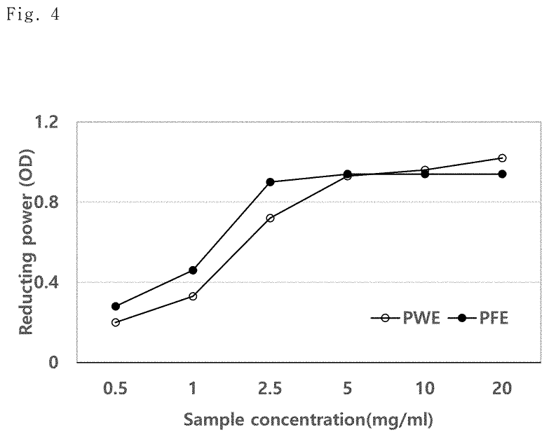

For Reducing power assay, the extracts of Example 1 were diluted to predetermined concentrations, and 1 mL of each dilution was added and reacted with 2.5 mL of 0.2 M phosphate buffer (pH 6.6) and 2.5 mL of 1% potassium ferricyanide at 50° C. for 30 minutes. Then 2.5 mL of 10% trichloroacetic acid was added, followed by centrifugation at 1,650×g for 10 minutes. To 2.5 mL of the supernatant thus obtained were 2.5 mL of distilled water and 0.5 mL of 0.1% FeCl₃ before reading absorbance at 700 nm on an ELISA reader (ECNSPIREMD, PerkinElmer, Germany). The results are summarized in Table 5 and depicted in FIG. 4.

TABLE 5

| | Prunus persica water | | | | | | Prunus persica prethanol | | | | | |
|---|---|---|---|---|---|---|---|---|---|---|---|---|
| | extract (mg/ml) | | | | | | extract (mg/ml) | | | | | |
| | 0.5 | 1 | 2.5 | 5 | 10 | 20 | 0.5 | 1 | 2.5 | 5 | 10 | 20 |
| Reducing | 0.20 | 0.33 | 0.72 | 0.93 | 0.96 | 1.02 | 0.28 | 0.46 | 0.90 | 0.94 | 0.94 | 0.94 |
| power (OD) | | | | | | | | | | | | |

As can be seen in Table 5 and FIG. 4, the extracts increased in reducing power in dose-dependent patterns. The capacity was 2- to 3-fold increased at a concentration of 2.5 mg/mL relative to 1 mg/mL, with the absorbance ranging from 0.72 to 0.90. The reducing power of the Prunus persica extracts was increased in a dose-dependent pattern from 0.2 to 1.02 for the Prunus persica leaf water extract and from 0.28 to 0.94 for the Prunus persica leaf prethanol extract.

### Example 6: Collagenase Inhibition Assay

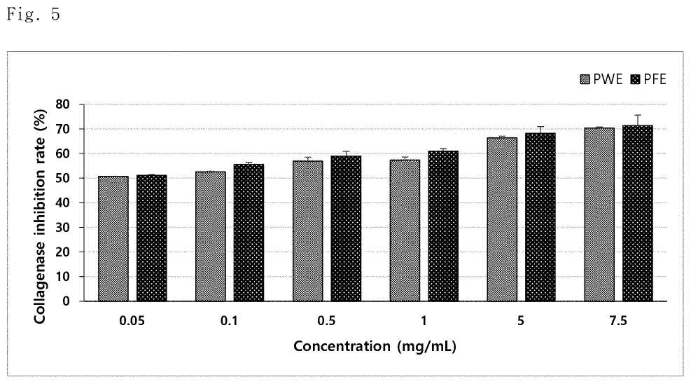

For reaction groups, 0.25 ml of a substrate solution containing 4-phenylazobenzyl oxycarbonyl Pro-Leu-Gly-Pro-D-Arg (0.3 mg/ml) dissolved in 0.1 M Tris-HCl buffer (pH 7.5) and 4 mM CaCl₂ was mixed with 0.1 ml of a sample and added with 0.15 ml of 0.2 mg/ml (Sigma-Aldrich Co.). For a control, 0.1 ml of distilled water was used instead of the sample. The mixtures were each left at room temperature for 20 min and added with 0.5 ml of 6% citric acid to terminate the reaction. After addition of 2 ml of ethyl acetate, absorbances were read at 320 nm and subjected to calculation according to Equation 3, below. The data are summarized in Table 6 and depicted in FIG. 5.

Collagenase Inhibition Rate (%)={1−(absorbance of sample added group/absorbance of non-added group)}×100 <Equation 3>

TABLE 6

| | Prunus persica water | | | | | | Prunus persica prethanol | | | | | |
|---|---|---|---|---|---|---|---|---|---|---|---|---|
| | extract (mg/ml) | | | | | | extract (mg/ml) | | | | | |
| | 0.05 | 0.1 | 0.5 | 1 | 5 | 7.5 | 0.05 | 0.1 | 0.5 | 1 | 5 | 7.5 |
| Collagenase | 50.66 | 52.55 | 56.91 | 57.31 | 66.32 | 70.32 | 51.08 | 55.52 | 58.88 | 60.91 | 68.17 | 71.32 |
| inhibition | | | | | | | | | | | | |
| rate (%) | | | | | | | | | | | | |

The extracts were measured for inhibitory activity against collagenase at concentrations of 0.05, 0.1, 0.5, 1, 5, and 7.5 mg/ml for each solvent. As can be seen in Table 6 and FIG. 5, the collagenase inhibition rate was observed to be 50.66% for the Prunus persica leaf water extract and 51.08% for the Prunus persica leaf prethanol extract at 0.05 mg/ml, and increased in a concentration-dependent pattern.

### Example 7: Tyrosinase Inhibition Assay

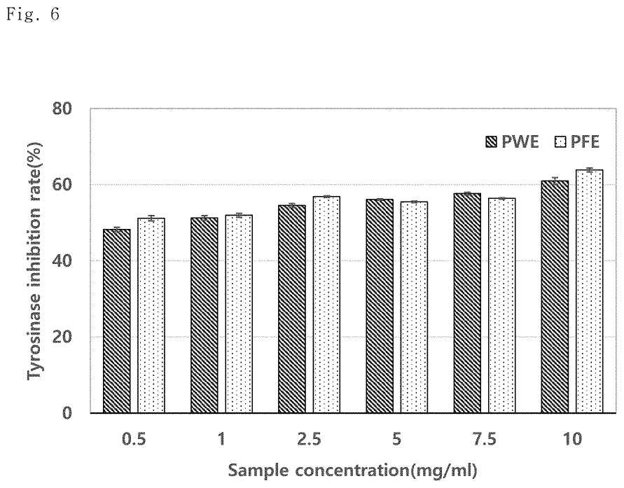

For tyrosinase inhibition assay, 0.4 mL of 1.5 mM L-tyrosine was added to 0.4 mL of 0.1 M sodium phosphate buffer (pH 6.0) and mixed with 0.2 mL of each of samples diluted to predetermined concentrations. The resulting solution was incubated with 0.1 mL of mushroom tyrosinase (100 U/mL) at 30° C. for 5 minutes. The DOPA chrome thus obtained was measured for absorbance at 475 nm using an ELISA reader (ECNSPIREMD, PerkinElmer, Germany). Distilled water was used instead of the enzyme solution or the sample. Tyrosine inhibition rate was calculated according to the following equation and the data are summarized in Table 7 and depicted in FIG. 6.

Tyrosinase Inhibition Rate (%)={1−(absorbance of sample-added group−absorbance of non-added group/absorbance of control)}×100 <Equation 4>

TABLE 7

| | Prunus persica water | | | | | | Prunus persica prethanol | | | | | |
|---|---|---|---|---|---|---|---|---|---|---|---|---|
| | extract (mg/ml) | | | | | | extract (mg/ml) | | | | | |
| | 0.5 | 1 | 2.5 | 5 | 7.5 | 10 | 0.5 | 1 | 2.5 | 5 | 7.5 | 10 |
| Tyrosinase | 48.23 | 51.25 | 54.51 | 56.08 | 57.65 | 60.94 | 51.18 | 51.96 | 56.86 | 55.49 | 56.39 | 63.84 |
| inhibition | | | | | | | | | | | | |
| rate (%) | | | | | | | | | | | | |

As can be seen in Table 7 and FIG. 6, the inhibition rate drastically increased in both the Prunus persica water extract and the Prunus persica prethanol ethanol until the concentration reached 2.5 mg/ml, and at higher concentrations, the inhibition rate was gradually increased. Particularly, at a concentration of 2.5 mg/ml, the inhibition rate was measured to be 54.51% for PWE and 56.86% for PFE. A slightly higher tyrosine inhibition rate was observed in the Prunus persica leaf prethanol extract than in the Prunus persica leaf water extract.

Phenolic compounds, flavonoids, arbutin, glycolic acid, kojic acid, and isoflavonoids are known as natural materials having tyrosinase inhibition activity. As for plants, their tyrosinase inhibition rates are known to be 42.00% for saltwort, 63.00% for morus bark, 13.00-52.00% for Glycyrrhiza uralensis, 44.00% for peony, 28.00% for Cnidium officinale, and 4.00% for Poria cocos Wolf. The peach leaf extracts in this study were observed to have higher inhibitory activity than the plants as measured for 48.23-57.65% in PWE and 27.84-56.86% in PFE.

### Example 8: Macrophage (Raw 264.7) Viability

Raw 264.7 cells and Fibroblast cells (Human Derma Fibrablast cells) were purchased from the Korean Cell Line Bank and grown in Dulbecco's modified Eagle's medium (Gibco, Co, USA) medium supplemented with 10% FBS, penicillin (100 μL/mL), and streptomycin (100 U/mL) at 37° C. under a 5% CO₂ condition. The RAW 264.7 cells were pretreated with various concentrations of the samples for 1 hour and then incubated with LPS for 24 hours.

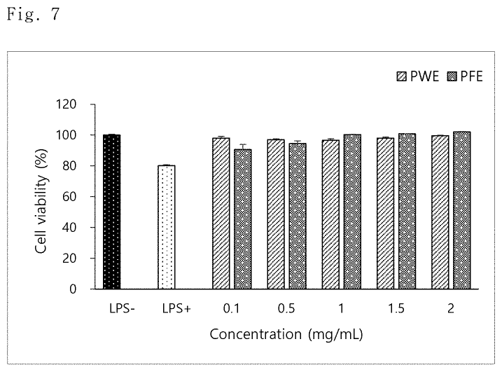

The Raw 264.7 was plated at a density of 5×10⁵ cells/mL into 96-well plates and the samples were added at various concentrations in an amount of 0.5 mL to each well before incubation at 37° C. for 24 hours in a 5% CO₂ incubator. For a control, distilled water was added in the same amount as the sample and incubated under the same condition. To each well were added 0.02 mL of 5 mg/mL MTT solution before incubation for 4 hours. The culture medium was aspirated, and 0.15 mL of DMSO:ethanol (1:1) was added to each well and incubated at room temperature for 30 minutes. Absorbance at 550 nm was read on an ELISA reader. Cell viability is expressed as % absorbance reduction of sample-added groups relative to non-added group according to Equation 5, below and the data are summarized in Table 8 and depicted in FIG. 7.

Cell viability (%)=[1−(absorbance of sample-added group/absorbance of non-added group)]×100 <Equation 5>

TABLE 8

| | | | Prunus persica water | | | | | Prunus persica prethanol | | | | |
|---|---|---|---|---|---|---|---|---|---|---|---|---|
| | | | extract (mg/ml) | | | | | extract (mg/ml) | | | | |
| | LPS− | LPS+ | 0.1 | 0.5 | 1 | 1.5 | 2 | 0.1 | 0.5 | 1 | 1.5 | 2 |
| Cell | 100.00 | 80.12 | 97.91 | 96.88 | 96.62 | 97.97 | 99.49 | 90.55 | 94.43 | 100.00 | 100.00 | 100.00 |
| viability | | | | | | | | | | | | |
| (%) | | | | | | | | | | | | |

As can be seen in Table 8 and FIG. 7, the cell viability was 80% or higher at each concentration (0.1, 0.5, 1, 1.5, and 2 mg/mL) of the Prunus persica leaf water extract and the Prunus persica leaf prethanol extract, indicating that each extract is free of cytotoxicity.

### Example 9: Nitric Oxide Inhibition Assay

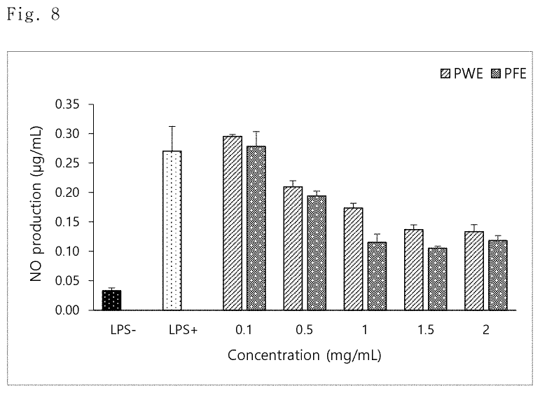

Macrophages were seeded at a density of 5×10⁵ cells/mL into 96-well plates and incubated for 24 hours. Then, the cells were treated with predetermined concentrations of the samples for one hour and incubated with 1 μg/mL LPS at 37° C. for 24 hours in a 5% CO₂ atmosphere. In the 96-well plates, a mixture of 100 μL of cell culture medium (DMEM) and 100 μL of Griess reagent [1% sulfanalamide+0.1% naphthylene-diamine dihydrochloride in 5% H₃PO₄ (1:1 mixture)] was added to each well and incubated at room temperature for 15 minutes. Optical density was measured at 540 nm on a microplate reader to quantitate NO. The data are summarized in Table 9 and depicted in FIG. 8.

TABLE 9

| | | | Prunus persica water | | | | | Prunus persica prethanol | | | | |
|---|---|---|---|---|---|---|---|---|---|---|---|---|
| | | | extract (mg/ml) | | | | | extract (mg/ml) | | | | |
| | LPS− | LPS+ | 0.1 | 0.5 | 1 | 1.5 | 2 | 0.1 | 0.5 | 1 | 1.5 | 2 |
| NO | 0.03 | 0.27 | 0.30 | 0.21 | 0.17 | 0.14 | 0.13 | 0.28 | 0.19 | 0.12 | 0.11 | 0.12 |
| production | | | | | | | | | | | | |
| (μg/ml) | | | | | | | | | | | | |

As can be seen in Table 9 and FIG. 8, No production was increased in the LPS group compared to the control (LPS−) whereas the groups treated with various concentrations of the Prunus persica leaf water extract and Prunus persica leaf prethanol extract significantly inhibited NO production. Particularly, NO production was inhibited to a higher extent in the Prunus persica leaf prethanol extract-treated groups than in the Prunus persica leaf water extract-treated groups.

### Example 10: Wound Healing Assay

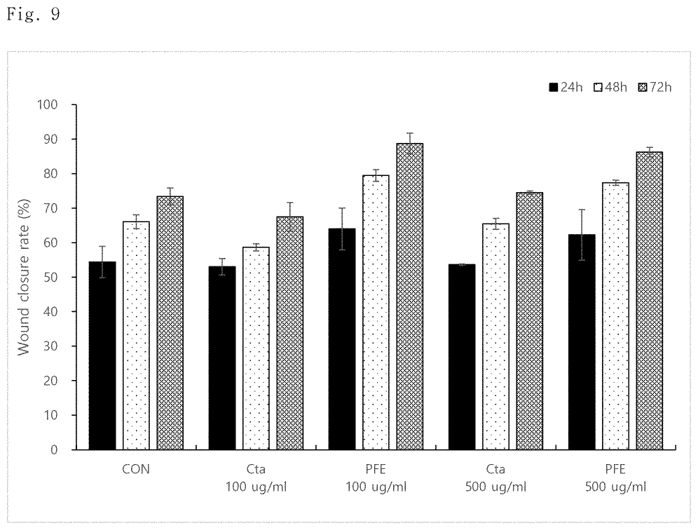

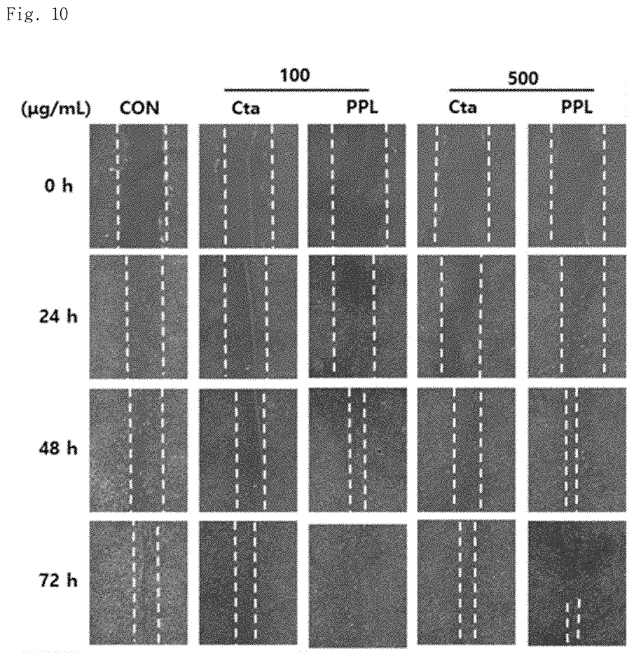

HDF cells were seeded at a density of 2×10⁵ cells/well into 12-well plates and incubated for 24 hours. The medium was changed with a FBS-free medium and scratches were made with a 200p tip on the well. The cells in each well were treated without (Con) or with an Centella asiatica extract (Cta) and the Prunus persica leaf prethanol extract (PFE) prepared in Example 1-2 at a concentration of 100 μg/ml and 500 μg/ml. Additional incubation was made for 24-72 hours after treatment with the substances. Then, the cells fixed by incubation with a fix solution (4% paraformaldehyde) for 15 minutes at room temperature, followed by washing three times with PBS. For the wound healing assay, microscopic images were analyzed using Image J software, and the data are summarized in Table 10 and depicted in FIGS. 9 to 10.

TABLE 10

| | | | Cta | PFE | Cta | PFE |
|---|---|---|---|---|---|---|
| | | | (100 | (100 | (500 | (500 |
| | | Con | μg/ml) | μg/ml) | μg/ml) | μg/ml) |
| Wound | 24 h | 54.38 | 53.05 | 64.00 | 53.65 | 62.25 |
| closure | 48 h | 66.07 | 58.05 | 79.48 | 65.46 | 77.36 |
| rate | 72 h | 73.40 | 67.49 | 88.74 | 74.48 | 86.22 |
| (%) | | | | | | |

As can be seen in Table 10 and FIGS. 9 and 10, the healing area was increased by 9.62-15.34%, compared to the control, upon treatment with the Prunus persica leaf extract at a concentration of 100 μg/ml. In addition, the healing area was increased by about 10.95-21.25%, compared to treatment with the positive control Centella asiatica extract.

### Example 11: Wound Healing Effect

The Prunus persica leaf extract prepared in Example 1 was examined for wound healing effect.

11-1. Experimental Group and Wound Induction

As experimental animals, male Sprague-Dawley 8 weeks old was maintained at a temperature of 21±2° C. and humidity of 55±10%, with 12-hours artificial lighting and allowed to freely access foods and water. After acclimation to the breeding room for one week, rats, each weighing 250 g, were randomly divided into groups of five. After being anesthetized with ether, the rats were shaved on the back and the shaved area was disinfected with iodine tincture. Four excisional wounds were made in a symmetrical pattern using a biopsy punch (8 mm). This experiment was approved by the Institutional Animal Care and Use Committee of Yeungnam University (YUMC-AEC2019-005).

The animals were classified into a normal control (NO) which was not treated, a positive control (MC) which was coated with Madecasol (DongKuk Pham, Seoul, Korea) after wound introduction, a coat group (PFE10) which was coated with 10 μL of the 1% Prunus persica leaf prethanol extract prepared in Example 1 after wound introduction, and a coat group (PFE100) which was coated with 100 μL of the 1% Prunus persica leaf prethanol extract after wound introduction, each group consisting of five rats. Until the scabs fell off, the coating solution was applied once a day in a sufficient amount to cover the wounded sites to minimize contact with air.

11-2. Observation of Wound Change with Naked Eye

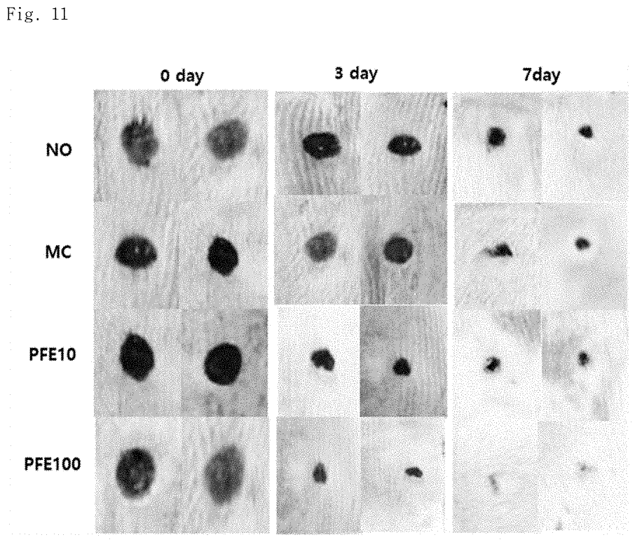

To monitor the procedure of wound healing, rats were randomly selected and anesthetized day 0, day 3, and day 7 after wound introduction and images including wound and normal sites were taken. The results are shown in FIG. 11.

As can be seen in FIG. 11, inflammation and exudation were observed in all the wound groups on the day of wound introduction. This acute inflammatory change was continued for 7 days after wound introduction. On day 3 following wound introduction, inflammatory responses were suppressed, with the consequent formation of scab, in PFE10 and PFE100 groups, compared to the other groups. PFE10 and PFE100 groups were identified to recover from the wound as their wound sizes were remarkably reduced, relative to those of the NO group and the control MC group. A severe wound is known to result in a scar. In this experiment, some scars were also observed on the wound sites on day 3 following wound introduction. However, the scars almost disappeared in observed PFE10 and PFE100 groups, compared to NO and MC groups. On day 7 following wound introduction, the wounds remained unclosed in the NO group while scars remained as a trace in the other groups. Inter alia, the wound sites were reconstituted into a normal epidermal form in the PFE100 group, with no clear boundary found between the wound and the normal tissue.

11-3. Histopathological Observation

Immunohistochemical Staining

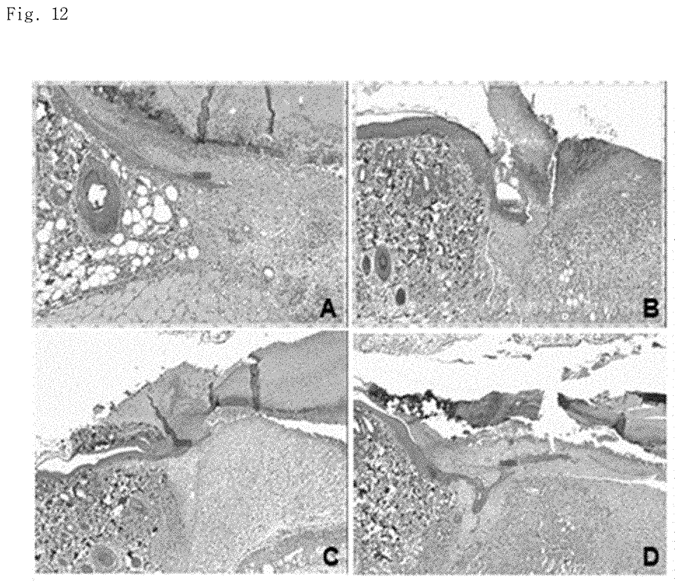

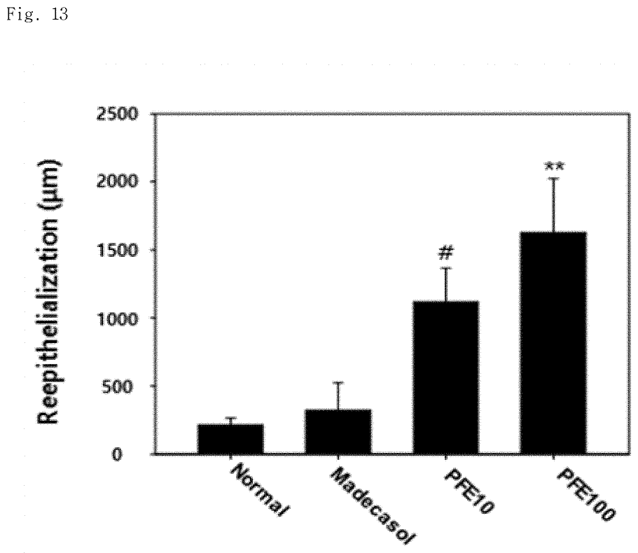

In order to examine epidermal regeneration following wound introduction, wounds were made with an 8-mm biopsy punch. Day 3 after application of each substance, rats in NO, MC, PFE10, and PFE100 groups were anesthetized. From the rats, tissues including both wound sites and normal skin were taken and fixed for 24 hours in a 10% neutral formalin solution. The tissues were examined with the naked eye and then embedded with paraffin and sectioned into 4 μm slices using an automated tissue processor. The sectioned tissues were dried at 60° C., deparaffinized, and hydrated. For quantitative analysis of the skin regeneration in the wound sites, immunohistochemical staining was conducted with an anti-rabbit pan-cytokeratin polyclonal antibody (dilution ratio 1:100, Bioss, Denmark). The tissues were treated with 3% hydrogen peroxide in methanol for 10 minutes so as to block endogenous peroxisase. For antigen revival, the slide was put in a citric acid buffer pH 6.0, autoclaved at 120° C. for 10 minutes, and cooled at room temperature for 20 minutes. After being washed with water, the slide was reacted with the primary antibody pan-cytokeratin antibody for 60 minutes and washed three times with a TBS buffer, each for 5 minutes. Then, the slide was reacted with the secondary antibody Dako EnVision+System-HRP Labelled polymer anti-rabbit kit for 30 minutes and washed three times with a TBS buffer, each for 5 minutes. Color development with DAB (3,3′-Diaminobenzidine) was followed by counter staining with Mayer hematoxylin. Thereafter, the tissues were washed, dehydrated, made transparent, and sealed. Using Aperio CS2 digital slide scanner (Leica Bio systems, USA), images were acquired. Degrees of staining in the regenerated epidermis were quantitatively measured by Aperio Image Scope (v 12.3.2.8013) (Leica Biosystems, USA) program, and the results are summarized in Table 11 and depicted in FIGS. 12 and 13.

TABLE 11

| | NO | MC | PFE10 | PFE100 |
|---|---|---|---|---|
| Reepithelialization | 213.30 | 322.20 | 416.60 | 784.50 |
| (μm) | | | | |

As can be seen in Table 11 and FIGS. 12 and 13, the immunohistochemical staining data showed that the epidermal regeneration was significantly increased in PFE10- and PFE100-coated groups, compared to the positive control MC.

Masson's Trichrome Stain

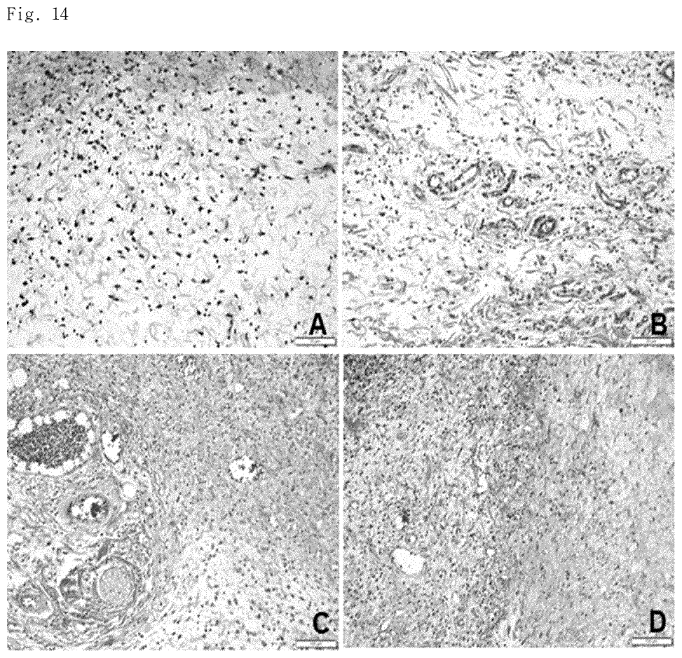

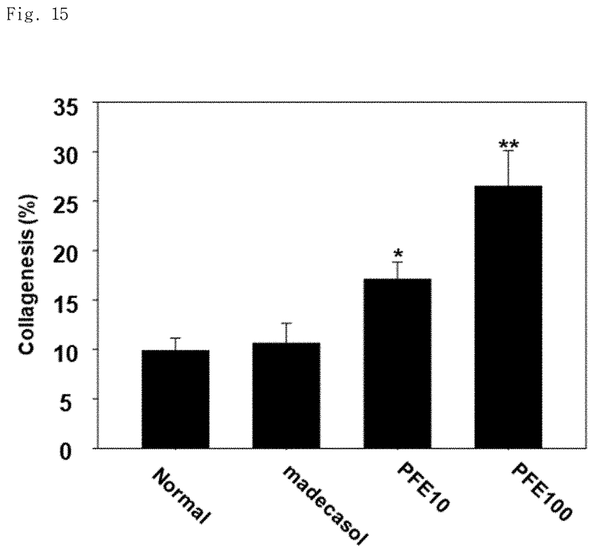

For collagen assay, rats in the NO, MC, PFE10, and PFE100 groups were anesthetized on day 3 after application of the substances. From the rats, tissues including both wound sites and normal skin were taken and fixed for 24 hours in a 10% neutral formalin solution. The tissues were examined with the naked eye and then embedded with paraffin and sectioned into 4 μm slices using an automated tissue processor. The sectioned tissues were dried at 60° C., deparaffinized, and hydrated. For quantitative analysis of collagen fibers, the tissues were stained for 10 minutes in a weight iron hematoxylin solution and for 15 minutes in a Biebrich scarlet red solution, followed by differentiation for 15 minutes with and phosphomolybdic acid/phosphotungstic acid. Then, staining for 15 minutes in aniline blue was followed by decoloration for 3 minutes in 1% acetic acid. Thereafter, the tissues were washed, dehydrated, made transparent, and sealed. Using DP71 microscope digital camera (Olympus, Japan), images were acquired. Collagen was quantitatively analyzed by Lepard (zootos, korea) program and the results are summarized in Table 12 and FIGS. 14 and 15.

TABLE 12

| | NO | MC | PFE10 | PFE100 |
|---|---|---|---|---|
| Collagenesis | 9.40 | 10.10 | 17.50 | 27.50 |
| (%) | | | | |

As can be seen in Table 12 and FIGS. 14 and 15, the Masson's Trichrome stain data showed that collagen generation was significantly increased in the PFE10 and PFE100 groups, compared to the positive control MC group.

### Example 12: Statistical Analysis

The data obtained in the Examples were subjected to analysis of variance using SPSS 17.0 (Statistical Package for Social Sciences, SPSS Inc., Chicago, Ill., USA). The Duncan's multiple range test was used at p<0.05 to establish significant differences among the treatments.

As described hitherto, the present disclosure relates to a composition comprising a Prunus persica extract for skin wrinkle reduction, anti-oxidation, skin regeneration, skin whitening, or wound healing, and a preparation method therefor and, more specifically, to a composition comprising a crude solvent extract of Prunus persica or a solvent fraction thereof as an active ingredient for skin wrinkle reduction antioxidation, skin regeneration, skin whitening, or wound healing and a preparation method therefor. A Prunus persica extract according to the present disclosure can be used in a cosmetic composition for skin wrinkle reduction, anti-aging through antioxidation, skin whitening, and skin regeneration or in a pharmaceutical composition useful for treatment of wounds.

What is claimed is:

1. A method for treating or relieving skin wound, the method comprising a step of:
applying to an affected area of a subject a composition comprising a Prunus persica extract as an active ingredient.

2. The method of claim 1, wherein the Prunus persica extract is an extract from leaves of Prunus persica.

3. The method of claim 1, wherein the Prunus persica extract is a crude extract obtained by extracting in at least one solvent selected from the group consisting of water, a straight or branched alcohol of 1 to 4 carbon atoms, and prethanol.

4. The method of claim 1, wherein the wound is at least one selected from the group consisting of a burn, a laceration, epidermal wound, ulcer, trauma, post-surgical wound, wound generated upon delivery, chronic wound, and injuries caused by dermatitis, corneal ulcer, corneal epithelial detachment, keratitis, and dry eye syndrome.

5. The method of claim 1, wherein the composition is formulated into an ointment encapsulated with a liposomal membrane.

6. The method of claim 3, wherein the solvent is an aqueous prethanol solution having an alcohol content of 20 to 40%.

7. A method for promoting skin regeneration, the method comprising a step of:
applying to a skin of a subject a composition comprising a Prunus persica extract as an active ingredient.

8. The method of claim 7, wherein the Prunus persica extract is a crude extract obtained by extracting in at least one solvent selected from the group consisting of water, a straight or branched alcohol of 1 to 4 carbon atoms, and prethanol.

9. The method of claim 8, wherein the solvent is an aqueous prethanol solution having an alcohol content of 20 to 40%.

10. The method of claim 7, wherein the composition is formulated into an ointment encapsulated with a liposomal membrane.

11. A method for cosmetically treating a skin condition, the method comprising a step of:
applying to a target skin a composition comprising a Prunus persica extract as an active ingredient.

12. The method of claim 11, wherein the Prunus persica extract is a crude extract obtained by extracting in at least one solvent selected from the group consisting of water, a straight or branched alcohol of 1 to 4 carbon atoms, and prethanol.

13. The method of claim 12, wherein the solvent is an aqueous prethanol solution having an alcohol content of 20 to 40%.

14. The method of claim 11, wherein the composition is formulated into an ointment encapsulated with a liposomal membrane.

15. The method of claim 11, wherein the Prunus persica extract is obtained from leaves of Prunus persica.
